(19) United States
(12) Patent Application Publication
(10) Pub. No.: US 2012/0328526 A1
(43) Pub. Date: Dec. 27, 2012

(54) Modulation of Nad+ Activity in Neuropathophysiological Conditions and Uses Thereof

(75) Inventor: Tibor Kristian, Severna Parks, MD (US)

(73) Assignee: University of Maryland, Baltimore

(21) Appl. No.: 13/533,686

(22) Filed: Jun. 26, 2012

Related U.S. Application Data
(60) Provisional application No. 61/501,280, filed on Jun. 27, 2011.

(51) Int. Cl. A61K 31/455; A61K 38/45; A61P 25/00; A61K 49/00; A61K 39/395; A61K 31/7088; A61K 38/00; A61P 25/02; A61K 31/706; A61P 25/28

(52) U.S. Cl. 424/9.2; 514/355; 514/43; 424/94.5; 424/158.1; 514/44 A; 514/17.7

(57) ABSTRACT

The present invention provides a method of treating a mammal having a neuropathophysiological condition, comprising the step of administering to the mammal in need of such treatment a compound selected from nicotinamide or salts or prodrugs thereof, nicotinamide mononucleotide or salts or prodrugs thereof, nicotinamide adenine dinucleotide or salts or prodrugs thereof, nicotinamide riboside nicotinamide or salts or prodrugs thereof, phosphoribosyltransferase, or combinations thereof. Further provided is a method for treating a mammal having a neuropathophysiological condition or suspected to develop said neuropathophysiological condition, comprising the step of administering to said mammal an inhibitor of CD38 NAD⁺ glycohydrolase activity.

### CROSS-REFERENCE TO RELATED APPLICATION

This non-provisional application claims benefit of priority under 35 U.S.C. §119(e) of provisional U.S. Ser. No: 61/501,280, filed Jun. 27, 2011, now abandoned, the entirety of which is hereby incorporated by reference.

### FEDERAL FUNDING LEGEND

This invention was made with government support under Grant Number NS058556 awarded by the National Institutes of Health. The government has certain rights in the invention.

### BACKGROUND OF THE INVENTION

1. Field of the Invention

The present invention is in the field of neurology and pharmacotherapy of neuropathophysiologic diseases and conditions. More specifically, the present invention is directed to modulation of NAD⁺ degradation in neuropathophysiologic diseases and conditions and uses thereof.

2. Description of the Related Art

It is becoming accepted that traumatic brain injury leads to chronic brain pathology that is associated with ongoing inflammation and persistent microglial activation. However, an efficient therapeutic approach to inhibit or at least reduce the chronic pathophysiology following traumatic brain injury, is elusive. The underlying pathophysiology of the chronic or the several month delayed brain structural abnormalities and pathologic cognitive functions following traumatic brain injury are poorly understood, although inflammation is potentially an important factor.

Approximately 30-40% of stroke patients are hyperglycemic as a result of diabetes (2). Type-2 diabetes holds a 2-6 fold increased risk for cerebrovascular disease, thus radically increasing stroke incidence in diabetic individuals (2). Moreover, hyperglycemia is associated with aggravated outcome in stroke (2). Despite extensive research, understanding of the mechanisms leading to poor outcome following brain ischemia is very limited.

There is evidence that physical injury produces a neuroinflammatory and excitotoxic response in the brain leading to an over-activation of microglia in the brain. Microglia react to this injury and become chronically activated. The clinical significance of persistent microglial activation, however, remains uncertain. In animal models, the initial inflammatory process can persist for at least a year. In humans, post-mortem studies have shown microglial activation many years after traumatic brain injury. Sites of activation coincide with those of neuronal degeneration and axonal abnormalities.

NAD⁺ is an important cofactor involved in multiple metabolic reactions having a central role in cellular metabolism and energy production (4). Uncontrolled PARP1 activation might deplete intracellular NAD⁺ and consequently ATP, leading to mitochondrial depolarization and cell death (8). Although it has been recognized that CD38 is a NAD³⁰ glycohydrolase, and has a role in the regulation of cellular NAD⁺ levels (8), there are no studies examining the contribution of this enzyme to NAD⁺ catabolism in neurodegenerative diseases.

CD38 is an ectoenzyme that uses NAD⁺ to generate cyclic ADP-ribose (cADPR) or ADP-ribose, and nicotinamide. Expression of CD38 in the brain can be found in specific populations of neurons, as well as astrocytes and microglia (3). Little is known about regulation of ADP-ribosyl cyclase or CD38 expression in neuronal cells. NAD³⁰ glycohydrolase activity was detected in isolated synaptosomes and in intact brain mitochondria (1), confirming localization of CD38 in mitochondrial membranes (10).

The distribution of NAD⁺ in cells and the locations of NAD⁺ synthesis have recently received consideration. NAD⁺ can be generated in cells either by de novo synthesis from tryptophan or it can be re-synthesized from nicotinamide (Nam) via a salvage pathway. Recently, a third vitamin precursor of NAD⁺ was discovered. Nicotinamide riboside (NR) is taken up by cells and phosphorylated to nicotinamide mononucleotide (NMN) by NR kinases (Nrk1 and Nrk2) (4). Nicotinamide mononucleotide is then adenylylated to form NAD⁺ by nicotinamide nucleotide adenylyltransferase (Nmnat) (4). Implied from the fact that Nmnat activity is required to complete all salvage and de novo pathways of NAD⁺ biosynthesis, mammalian cell NAD⁺ is compartmentalized. Cellular fractionation studies have shown that mitochondria maintain relatively high NAD⁺ concentrations and that mitochondrial NAD⁺ does not readily leak across the inner membrane. The efficiency of de novo and salvage pathways seems to be higher in glia compared to neurons (11).

Nicotinamide mononucleotide and the recently discovered nicotinamine riboside are alternative precursors for NAD⁺ biosynthesis that are utilized by the NAD⁺ salvage pathway.

Application of NMN leads to increases in cellular NAD⁺ levels by a one-step enzymatic reaction where NMN is converted to NAD⁺ by Nmnat (4). NMN may also inhibit CD38 NAD⁺glycohydrolase activity, reducing NAD⁺ and ATP depletion in cells undergoing PARP1 hyperactivation and significantly delaying cell death (12).

The prior art is deficient in methods of treatment of neuropathophyiological conditions such as ischemic injury. The present invention fulfills this longstanding need and desire in the art.

### SUMMARY OF THE INVENTION

The present invention is directed to a method of treating a mammal having a neuropathophysiological condition, comprising the step of administering to the mammal in need of such treatment a compound selected from nicotinamide or salts or prodrugs thereof, nicotinamide mononucleotide or salts or prodrugs thereof, nicotinamide adenine dinucleotide or salts or prodrugs thereof, nicotinamide riboside or salts or prodrugs thereof, phosphoribosyltransferase (NAMPT), or combinations thereof.

The present invention is further directed to a method for treating a mammal having a neuropathophysiological condition or suspected to develop said neuropathophysiological condition, comprising the step of: administering to said mammal an inhibitor of CD38 NAD⁺glycohydrolase activity.

The present invention is further directed to a method for identifying an agent for treating a neuropathophysiological condition, wherein said method comprises: (a) determining whether or not a test agent inhibits CD38, wherein inhibition of CD38 indicates that said test agent is a candidate agent, and (b) administering said candidate agent to a mammal to determine whether or not said candidate agent treats the neuropathophysiological condition in said mammal, wherein an improvement in symptoms of said neuropathophysiological condition indicates that said candidate agent is said treatment agent.

### BRIEF DESCRIPTION OF THE DRAWINGS

The appended drawings have been included herein so that the above-recited features, advantages and objects of the invention will become clear and can be understood in detail. These drawings form a part of the specification. It is to be noted, however, that the appended drawings illustrate preferred embodiments of the invention and should not be considered to limit the scope of the invention.

FIGS. 1A-1B show effects of nicotinamide mononucleotide administration. FIG. 1A: Fluoro-Jade B staining of the hippocampal formation of mouse brain subjected to 10 min forebrain ischemia and 5 days of recovery. The middle panels represent enlarged views of the CA1 sector, the CA2-3 border, and DG. Note the high number of degenerating, Fluoro-Jade B positive cells in the CA1 pyramidal cell layer. There are only a few cells stained in the CA3 region, and some cells are stained in the DG. FIG. 1B: Dramatic reduction of ischemia-induced brain damage by NMN administration. Histological outcome in the CA1 sector of the hippocampus after 3 (n=6) and 5 (n=6) days recovery or, at 5 days for those post-treated with 500 mg/kg NMN (n=6). *p<0.01, when compared to sham. Open bars represent average values.

FIGS. 2A-2C show Cresyl-Violet and lba1 immunostaining of the hippocampal formation (DG subregion) of diabetic mouse brain subjected to 4 min forebrain ischemia. FIG. 2A: Ramified microglia with small bodies and abundant processes (control, DG). FIG. 2B: Hypertrophic microglia with thicker processes appear before pathologic alterations in neuronal histology (Cresyl-Violet staining, 24 h recovery). FIG. 2C: Activated microglia (large irregular bodies with few processes, 2 days recovery). Black arrow heads—microglia, white arrow head dead neurons with dark shrunken cell bodies.

FIGS. 3A-3B show the effect of forebrain ischemia and nicotinamide mononucleotide administration on tissue NAD(H) levels. Mice were subjected to 10 min transient forebrain ischemia and allowed to recover for 24 h. After decapitation their brains were removed, the designated regions dissected and frozen in liquid nitrogen. One group of animals received nicotinamide mononucleotide at 1 hour of recovery. The NAD content was determined following perchloric acid extraction and utilized a coupled fluorometric cycling enzymatic assay (FIG. 3A). There is significant loss of NAD⁺ in striatum, CA1, and DG regions. *p<0.05. The NMN treatment prevented the post-ischemic NAD catabolism. Black: sham; white: ischemia; gray: NMN treatment. FIG. 3B: Effect of ischemia on CD38 activity. Brains of sham operated animals or animals subjected to 10 min of forebrain ischemia and 24 h of recovery were used for dissection of designated brain regions and homogenized. The CD38 enzyme activity was determined using 1-etheno-NAD that, when hydrolyzed by CD38, generates a fluorescent product. The rate of fluorescence increase corresponds to the CD38 enzyme activity. Blue: sham (n=4), red: ischemic samples (n=4). *p<0.05. FIGS. 4A-4B show effects in post-ischemic hippocampal tissue. FIG. 4A: Inhibition of maximal respiratory capacity of synaptic mitochondria isolated from post-ischemic hippocampal tissue (bottom trace). Top trace represents the oxygen consumption rates of control mitochondria. FCCP—uncoupler; stimulates maximal respiration, Antimycin—inhibitor of mitochondrial respiration. FIG. 4B: NAD(H) levels in control and post-ischemic hippocampal mitochohdria and synaptosomes.

FIG. 5 shows that mitochondria show CD38 activity. Addition of isolated brain mitochondria into medium containing 1-etheno-NAD (eNAD) results in generation of fluorescent product. This NAD catabolism is inhibited by nicotinamide mononucleotide (1 mM). FIG. 6 shows CD38 expression is markedly increased following ischemic insult. In brain tissue of sham-operated animals the CD38 expression is very low. However, at 24 h of recovery following ischemic insult the CD38 levels are strikingly increased in vulnerable hippocampal areas. This increase is observed particularly in non-neuronal cells that resemble post-ischemic microglia.

FIG. 7 shows that CD38 is not present in CD38−/− mouse. WT: sample of hippocampus from wild type animal. CD38−/−: hippocampus from CD38 knockout. Spleen: positive control from WT mouse spleen tissue.

FIG. 8 shows neuronal damage in the CA1 sector of the hippocampus in control (sham operated mice), in wild type animals treated with vehicle or NMN and in CD38 knockout animals after ischemic insult. isch, vehicle—vehicle treated group; isch, NMN-nicotinamide mononucleotide treated group (1000 mg/kg at the time of start of reperfusion IP); isch, CD38KO-CD38 knockout animals. *p<0.05; **p<0.01 when compared to isch, vehicle group. n=4-6. In all groups male, 3 months old animals were used.

FIGS. 9A-9B show traumatic brain injury-induced deficits in sensorimotor function and cognition are ameliorated by nicotinamide mononucleotide treatment. FIG. 9A: Fine motor coordination deficits were quantified using a beam walk test in C57Bl/6 mice subjected to moderate-level traumatic brain injury and treated with nicotinamide mononucleotide (500 mg/kg, I.P.) or equal volume of vehicle at 1 hour post-injury. Hindlimb foot placement was recorded up to 7 days post-injury and the number of foot faults recorded from 50 steps. Naïve C57Bl/6 mice served as a control group. NMN treatment resulted in significantly reduced deficits in fine motor coordination at 7 days post-injury when compared to vehicle-treated traumatic brain injury mice (p<0.001). Analysis was by repeated measures one-way ANOVA, followed by post-hoc adjustments using Student-Newman-Keuls test. Mean ±S.E.M.; n=5/group. FIG. 9B: Cognitive performance was analyzed by measuring the number of anti-clockwise and clockwise rotations of traumatic brain injury mice in an open field test performed at 7 days post-injury. TBI induced significant impairments in cognitive performance demonstrated by increased anti-clockwise rotations in the open field test. In contrast, NMN-treated traumatic brain injury mice had reduced anti-clockwise rotations and were indistinguishable from naïve non-injured control mice. **p<0.01 when compared to the traumatic brain injury group.

### DETAILED DESCRIPTION OF THE PRESENT INVENTION

As used herein, the term “a” or “an”, when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, method steps, and/or methods of the invention. It is contemplated that any method, compound, composition, or device described herein can be implemented with respect to any other device, compound, composition, or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the term “about” refers to a numeric value, including, for example, whole numbers, fractions, and percentages, whether or not explicitly indicated. The term “about” generally refers to a range of numerical values, e.g., +/−5-10% of the recited value, that one of ordinary skill in the art would consider equivalent to the recited value, e.g., having the same function or result. In some instances, the term “about” may include numerical values that are rounded to the nearest significant figure.

Treatment of a variety of neuropathophysiological conditions such as neurologic diseases and ischemic injury are elusive. The present invention demonstrates that a CD38-dependent NAD⁺ degradation and microglial overactivation significantly contribute as aggravating factors to ischemic brain injury. Unraveling the glial-specific and neuron-specific role of the NAD⁺ glycohydrolase CD38 in mechanisms of NAD⁺ depletion offers new therapeutic targets for the treatment of ischemic injury. Finally, establishing the extraordinary protective effect of nicotinamide mononucleotide and that nicotinamide riboside protects against ischemic brain damage in subjects, will have a significant impact on the clinical application of these NAD⁺ precursors as therapeutic compounds for acute or chronic neurodegenerative disease. One can utilize several strategies to inhibit the reduction in cellular NAD⁺ levels during pathologic conditions. One approach being to maintain cellular NAD⁺ levels following an ischemic insult by administration of NAD⁺ precursors to facilitate NAD⁺ generation by the salvage pathway.

Thus, in one embodiment of the present invention, there is provided a method of treating a mammal having a neuropathophysiological condition, comprising the step of administering to the mammal in need of such treatment a compound selected from nicotinamide or salts or prodrugs thereof, nicotinamide mononucleotide or salts or prodrugs thereof, nicotinamide adenine dinucleotide or salts or prodrugs thereof, nicotinamide riboside or salts or prodrugs thereof, phosphoribosyltransferase, or combinations thereof. For example, the compound may be nicotinamide or salts or prodrugs thereof, nicotinamide mononucleotide, nicotinamide adenine dinucleotide, nicotinamide phosphoribosyltransferase, or combinations thereof. Alternately, the compound is a polypeptide capable of catalyzing the conversion of nicotinamide to nicotinamide mononucleotide. Representative conditions which may be treated using this method include but are not limited to traumatic brain injury, ischemia, stroke, Alzheimer's Disease, multiple sclerosis, amyotrophyc lateral sclerosis and peripheral neuropathy. Nicotinamide mononucleotide may be administered in a dose of from about 10 mg/kg to about 1000 mg/kg. In a preferred embodiment, the mammal is a human.

In another embodiment of the present invention, there is provided a method for treating a mammal having a neuropathophysiological condition or suspected to develop said neuropathophysiological condition, comprising the step of administering to said mammal an inhibitor of CD38 NAD⁺ glycohydrolase activity. A person having ordinary skill in this art would readily recognize that there are a variety of techniques to modulate, regulate or inhibit the activity of CD38 NAD⁺ glycohydrolase. A representative inhibitor is an anti-CD38 antibody or flavonoids such as luteolinidin, kuromanin and luteolin (13). Alternately, an inhibitor of CD38 NAD⁺ glycohydrolase is a compound selected from nicotinamide or salts or prodrugs thereof, nicotinamide mononucleotide or salts or prodrugs thereof, nicotinamide adenine dinucleotide or salts or prodrugs thereof, nicotinamide riboside nicotinamide or salts or prodrugs thereof, nicotinamide phosphoribosyltransferase, or combinations thereof. Representative conditions which may be treated using this method include but are not limited to traumatic brain injury, ischemia, Alzheimer's Disease, multiple sclerosis, amyotrophyc lateral sclerosis and peripheral neuropathy. Nicotinamide mononucleotide may be administered in a dose of from about 10 mg/kg to about 1000 mg/kg. In a preferred embodiment, the mammal is a human.

In yet another embodiment of the present invention, there is provided a method for identifying an agent useful for treating a neuropathophysiological condition, wherein the method comprises: a) determining whether or not a test agent inhibits CD38, wherein inhibition of CD38 indicates that the test agent is a candidate agent, and b) administering the candidate agent to a mammal to determine whether or not the candidate agent treats the neuropathophysiological condition in the mammal, wherein an improvement in symptoms of the neuropathophysiological condition indicates that said candidate agent is the treatment agent. In one aspect, this method comprises using an in vitro CD38 activity assay. A representative mammal is a mouse.

As described above, an inhibitor of CD38 can be used to treat neuropathophysiological conditions in a mammal. The mammal can be any type of mammal use in a human is a preferred embodiment. An inhibitor of CD38 can be any agent that reduces CD38 expression, e.g., an siRNA molecule, antisense oligonucleotide, or peptide nucleic acid, or CD38 activity, e.g., an inhibitory anti-CD38 antibody or CD38 antagonist such as nicotinamide or nicotinic acid.

Agents that can inhibit CD38 expression or activity in cells can be identified by screening candidate agents, e.g., from synthetic compound libraries and/or natural product libraries. Candidate agents can be obtained from any commercial source and can be chemically synthesized using methods that are known to those of skill in the art. Candidate agents can be screened and characterized using in vitro cell-based assays, cell free assays, and/or in vivo animal models. For example, a CD38 NADase assay can be used to identify CD38 antagonists. NADase activity can be determined using etheno-NAD. Enzyme preparations, e.g., purified CD38, can be incubated in a medium containing, for example, 0.2 mM NGD, 0.25 M sucrose, and 40 mM Tris-HCl (pH 7.2) at 37° C. NADase activity can be determined by measuring the change in fluorescence over time at, for example, 300 nm excitation and 410 nm emission. Candidate agents can be added to the CD38 assay, and a decrease of the NADase activity compared to control can be determined. A reduction in NADase activity can indicate that the candidate agent is a CD38 antagonist and can be used to treat neuropathophysiological conditions as described herein.

An inhibitor of CD38 can be administered to a mammal alone or in combination with other agents such as another inhibitor of CD38. For example, a composition containing an anti-CD38 antibody can be administered to a mammal in need of treatment. Such a composition can contain additional ingredients including, without limitation, pharmaceutically acceptable vehicles. A pharmaceutically acceptable vehicle can be, for example, saline, water, lactic acid, or mannitol.

A composition containing an inhibitor of CD38 can be administered to mammals by any appropriate route, such as enterally, e.g., orally, parenterally, e.g., subcutaneously, intravenously, intradermally, intramuscularly, or intraperitoneally, intracerebrally, e.g., intraventricularly, intrathecally, or intracisternally, or intranasally, e.g., by intranasal inhalation. Suitable formulations for oral administration can include tablets or capsules prepared by conventional means with pharmaceutically acceptable excipients such as binding agents, e.g., pregelatinized maize starch, polyvinylpyrrolidone or hydroxypropyl methylcellulose, fillers, e.g., lactose, microcrystalline cellulose or calcium hydrogen phosphate, lubricants, e.g., magnesium stearate, talc or silica, disintegrants, e.g., potato starch or sodium starch glycolate, or wetting agents, e.g., sodium lauryl sulfate. Tablets can be coated by methods known in the art. Preparations for oral administration can also be formulated to give controlled release of the agent.

Intranasal preparations can be presented in a liquid form, e.g., nasal drops or aerosols, or as a dry product, e.g., a powder. Both liquid and dry nasal preparations can be administered using a suitable inhalation device. Nebulized aqueous suspensions or solutions can also be prepared with or without a suitable pH and/or tonicity adjustment.

A composition containing an inhibitor of CD38 can be administered to a mammal in any amount, at any frequency, and for any duration effective to achieve a desired outcome, e.g., to treat a neuropathophysiological condition. An effective amount of an inhibitor of CD38 can be any amount that treats a mammal's neuropathophysiological conditions without producing significant toxicity to a mammal. Typically, an effective amount of an inhibitor of CD38 can be any amount greater than or equal to about 10 .mu.g provided that that amount does not induce significant toxicity to the mammal upon administration. In some cases, an effective amount of an inhibitor of CD38 can be between 1 .mu.g and 500 mg, e.g., between 1 .mu.g and 250 mg, between 1 .mu.g and 200 mg, between 1 .mu.g and 150 mg, between 1 .mu.g and 100 mg, between 1 .mu.g and 50 mg, between 1 .mu.g and 10 mg, between 1 .mu.g and 1 mg, between 1 .mu.g and 100 .mu.g, between 1 .mu.g and 50 .mu.g, between 5 .mu.g and 100 mg, between 10 .mu.g and 100 mg, between 100 .mu.g and 100 mg, or between 10 .mu.g and 10 mg. Various factors can influence the actual effective amount used for a particular application. For example, the frequency of administration, duration of treatment, use of multiple treatment agents, route of administration, and severity of the neuropathophysiological condition may require an increase or decrease in the actual effective amount administered.

This document also provides methods and materials for identifying agents that can be used to treat a mammal having or being likely to treat a neuropathophysiological condition. For example, a CD38 activity assay (e.g., a CD38 NADase assay) can be used to identify agents that can be used to treat a mammal having or being likely to develop a neuropathophysiological condition.

The following example(s) are given for the purpose of illustrating various embodiments of the invention and are not meant to limit the present invention in any fashion.

### EXAMPLE 1

### Forebrain Ischemia Model

C57BL/6 male mice, weighing 20-25 g were used. Mice were fasted overnight with free access to water ad libitum. Animals were anesthetized with 3.5% isoflurane in 70% N₂O and 30% O₂ and the trachea were intubated with a 20-guage intravenous catheter. Anesthesia was maintained using 1.5% isoflurane in N₂O:O₂ (70:30) and the lungs were mechanically ventilated at a rate of 120 breaths per min and the tidal volume was adjusted to give normal physiological values of blood gases (34-38 mm Hg pCO₂ and 10-120 mm Hg of pO₂). The common carotid arteries was isolated via a neck incision and encircled with loose ligatures for later clamping. Rectal temperature was routinely monitored before, during and after ischemia. A subcutaneous needle thermistor was placed adjacent to the skull. The body and head temperature was maintained at 37.0±0.5° C. by a homeothermic-heating pad and heating lamp.

Two minutes before the common carotid arteries are clamped, the isoflurane concentration will be increased to 5%. Following a 2 min period, the mean arterial blood pressure drops to 40-50 mm Hg. At this time, both common carotid arteries were occluded with aneurysm clips. After clamping, the mean arterial blood pressure stabilizes at 30-35 mm Hg. The common carotid arteries were occluded for 4 minutes. Four minutes before the clips were removed the anesthesia is discontinued by reducing the isoflurane concentration to 0%. At the time of clip removal, the mean arterial blood pressure reaches a level of 40-50 mm Hg. Then the wound was closed with sutures. The trachea was extubated upon recovery of the righting reflex and temperature regulation was discontinued. The mice were then moved into pre-warmed cages to prevent post-ischemic hypothermia.

### EXAMPLE 2

### Stereologic Quantifications

Every sixth 30 mm-thick section located between 1.7 and 2.6 mm posterior to bregma (0 mm, 180 mm, 360 mm, 540 mm, 720 mm, 900 mm; 6 sections), which comprises the dorsal hippocampus, was stained with IBA1 antiserum and cresyl violet and used for estimating the total microglial number with distinct morphology and neuronal numbers of normal and dying neurons in the different hippocampal regions. Microglial cells were classified into three categories, based on visual inspection. Parallel sections were immuno-stained with GFAP antibody and the number of distinct astrocytes were counted in the same hippocampal area. GFAP-immunoreactive cells were classified into three categories: normal, moderate, and intensely activated astrocytes, based on morphological characteristics as assessed by visual inspection (23).

Quantitative analysis is performed on a computer-assisted image analysis system consisting of a Nikon Eclipse 800 photomicroscope equipped with a computer-controlled motorized stage, and a computer running Stereolnvestigator program, a custom-designed morphology and stereology software (27). Cell numbers were quantified according to the optical fractionator method. The stage-controlling computer randomly places the counting frame on the first counting area, then systematically moves it until the entire delineated field is sampled. For cresyl violet staining, dying neurons were defined as those exhibiting either clear accumulation of dense, globular materials in the cytoplasm with evidence of nuclear fragmentation, or cells showing shrunken perikarya and darkly stained nuclei of reduced size (27).

### EXAMPLE 3

Neurologic outcome

Following recovery intervals of 3 and 7 days, the mice were subjected to a neurologic examination designed to detect motor and cognitive deficits (23). Three different tests for motor deficiency were used. The first tests the ability of the mouse to hold onto a screen after being rotated from a horizontal to a vertical position. The next test evaluates the amount of time that the mouse can remain balanced on a horizontal rod. Lastly, the mouse undergoes a prehensile traction test by timing the period that it can cling to a horizontal rope. All three tests were scored, and the sum of these scores represents the motor ability of individual animals.

### EXAMPLE 4

### Histological Outcome

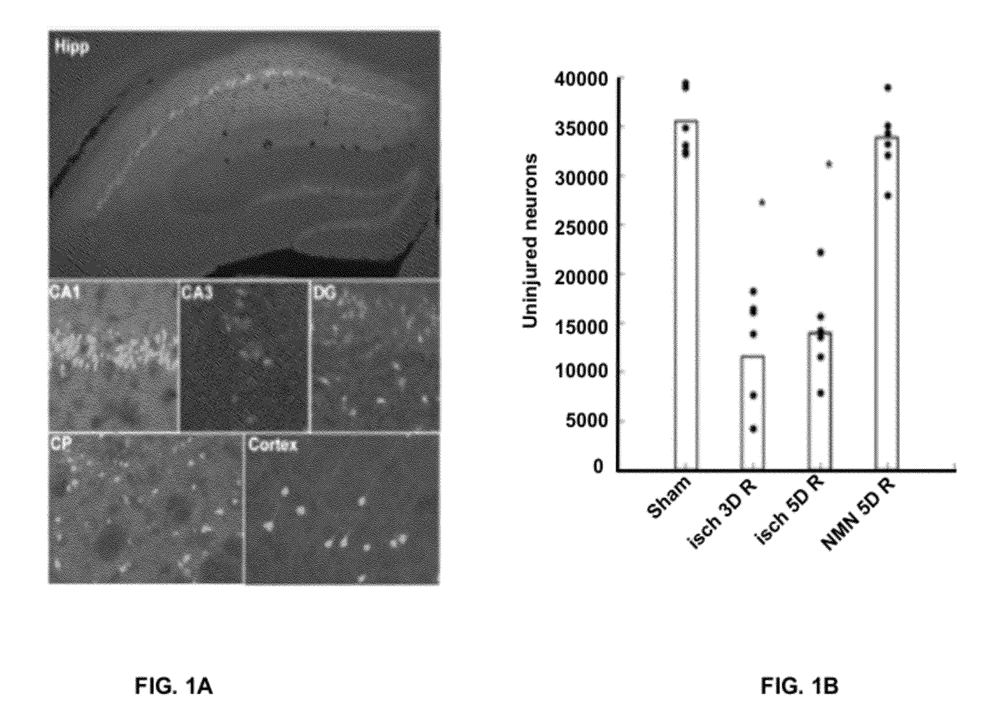

Mice subjected to 10 min forebrain ischemia underwent 3 or 5 days of recovery. On the last day of recovery, the animals were perfusion-fixed. Their brains were cut and processed for Fluoro-Jade B. As FIG. 1A shows, there is an intense and dense staining of degenerative cells in the CA1 sector of the hippocampus. Interestingly, some cells in the DG region were also positive for Fluoro-Jade B staining. The Fluoro-Jade B staining also revealed neurodegeneration in the striatum and in upper layers of the parietal cortex (FIG. 1A, bottom panel).

For quantification of uninjured cells within the CA1 sector of the hippocampus an unbiased stereological technique was used (27). As FIG. 1B shows, at 3 and 5 days of recovery there was about 60% cell death in the CA1 and was not significantly different between 3 and 5 days of reperfusion. This is in accord with the degree of hippocampal injury reported in other models of mouse forebrain ischemia. There were no seizure incidents during the first hours of recovery however there was about 10% mortality in the 5 days recovery group. Remarkably, after administration of nicotinamide mononucleotide (NMN, 500 mg/kg) at 1 hour of reperfusion the cell death at 5 days of recovery was almost completely ameliorated. Five of 6 animals did not show any injured neurons. To exclude the possibility that the protection was due to hypothermia, the treated animals' core temperature was monitored for 1 h after the nicotinamide mononucleotide administration. The body temperature was stable at 37.5° C. Similarly there was no effect of nicotinamide mononucleotide administration on the animals' blood pressure that was maintained between 80 and 100 mmHg after the compound injection.

### EXAMPLE 5

### Global Cerebral Ischemia in Diabetic Animals

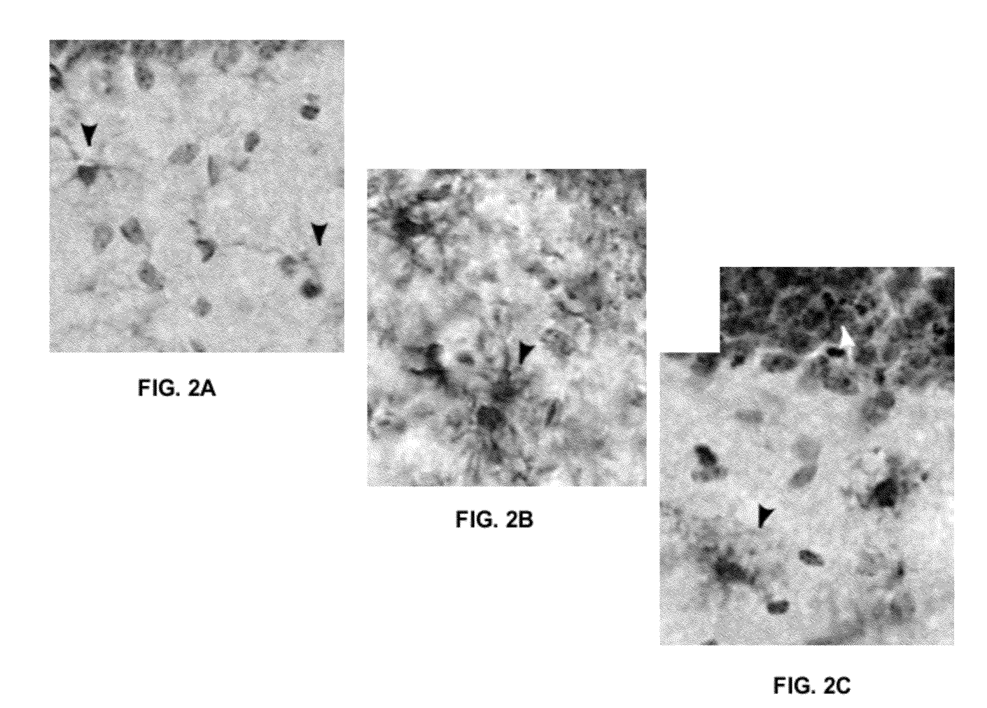

One month after the animals were made diabetic by streptozotocin injection, they were subjected to different periods of forebrain ischemia. When the ischemic period was reduced from 10 to 4 min the animals survived more then 3 days. A five min or longer period of ischemia resulted in animals' death within 1-2 days of recovery. lba1 immunostaining revealed, compared to control (FIG. 2A), robust microglial activation and their migration into the pyramidal cellular layer when neuronal death is manifested at 2 days of reperfusion (FIG. 2C, irregular amoeboid body with few short processes). However, hypertrophic and “bushy” microglial morphology (large irregular bodies with thicker processes) appeared after 24 hours of recovery when no gross morphological alterations indicating cell death had occurred (FIG. 2B). This early post-insult transformation of microglia was observed only in diabetic animals. These data show that post-treatment of animals with nicotinamide mononucleotide markedly ameliorates ischemic brain damage.

### EXAMPLE 6

### NAD⁺ Catabolism, Mitochondrial Function, and CD38 Activity in Post-ischemic Brain

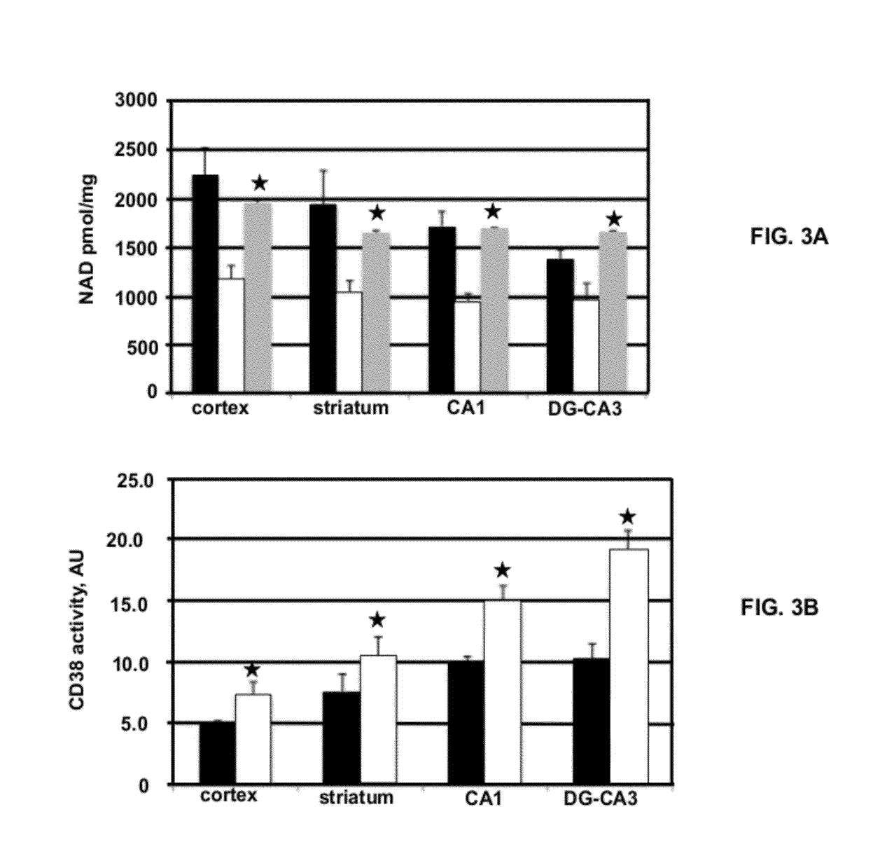

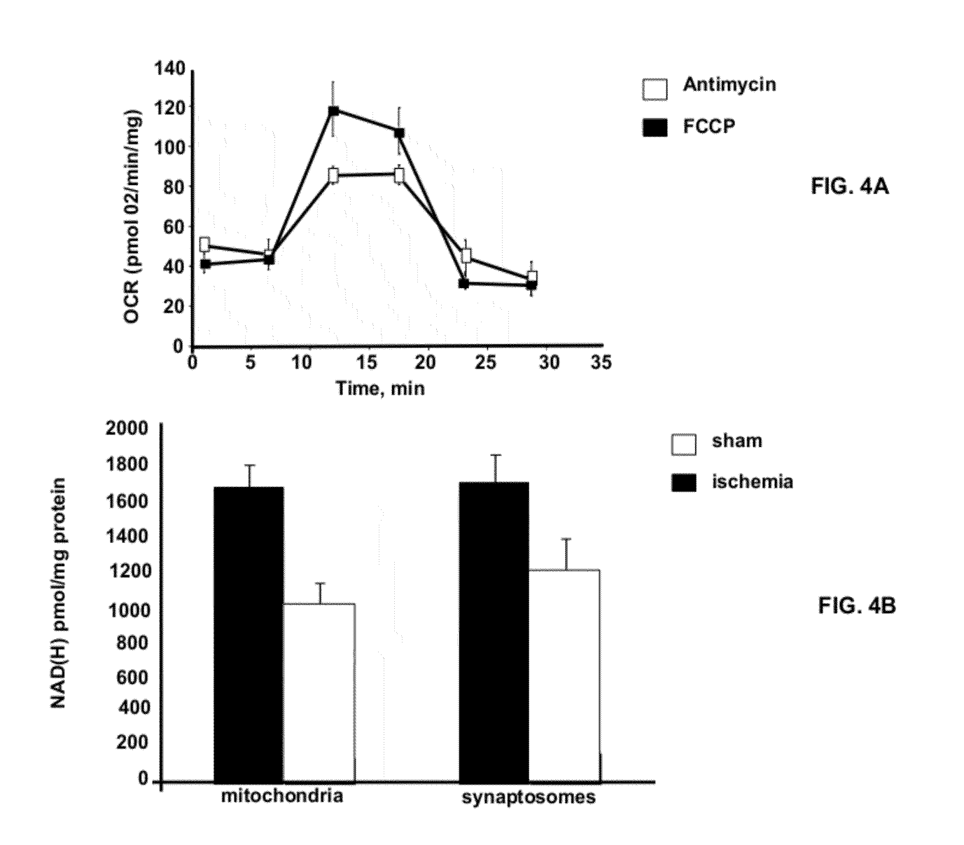

Since the first gross morphological alterations resulting from cell death were observed at 48 hours of reperfusion, the biochemical changes in designated brain regions were determined at 24 hour after the ischemic insult before the imminent cell death. The tissue NAD(H) levels were determined utilizing an enzymatic cycling assay. Control sham operated animals and animals after 24 hours of recovery following forebrain ischemia were decapitated and their brains removed and dissected to assess tissue NAD(H). The parietal cortex, striatum, the CA1 sector and CA3-DG regions of the hippocampus were dissected and the tissue NAD(H) levels were determined following perchloric acid extraction. As FIG. 3A demonstrates, the NAD(H) levels were significantly reduced in all brain regions. In the hippocampal CA1 region, the reduction was more than 50%. The profound reduction in brain NAD(H) levels in post-ischemic animals was reversed in mice treated with nicotinamide mononucleotide. Nicotinamide mononucleotide was administrated intraperitoneally at 1 h of reperfusion and the tissue NAD(H) content was determined at 24 h of recovery.

Since nicotinamide mononucleotide is inhibiting the CD38 NAD⁺ glycohydrolase activity, the activity of CD38 in post-ischemic tissue was assessed. The NAD⁺ glycohydrolase activity was measured by monitoring the enhancement in fluorescence emission caused by the hydrolysis of 1-etheno-NAD (eNAD) (9). The individual brain regions were homogenized and centrifuged at low speed to sediment the unbroken cells and the nuclear fraction. Aliquots of the collected supernatant were used to determine the CD38 activity. The NAD⁺ glycohydrolase activity was significantly elevated at 24 h of recovery in all brain subregions when compared to sham operated animals (FIG. 3B). This eNAD hydrolysis was completely inhibited in the presence of nicotinamide mononucleotide (1 mM). Since the samples are free of the nuclear fraction and are enriched by synaptosomes and mitochondria, contribution of PARP-1 to the eNAD degradation in the suspension is negligible. Furthermore, the specificity of eNAD degradation by CD38 was confirmed by using samples from CD38-null animals that show negligible e-NAD hydrolysis (6).

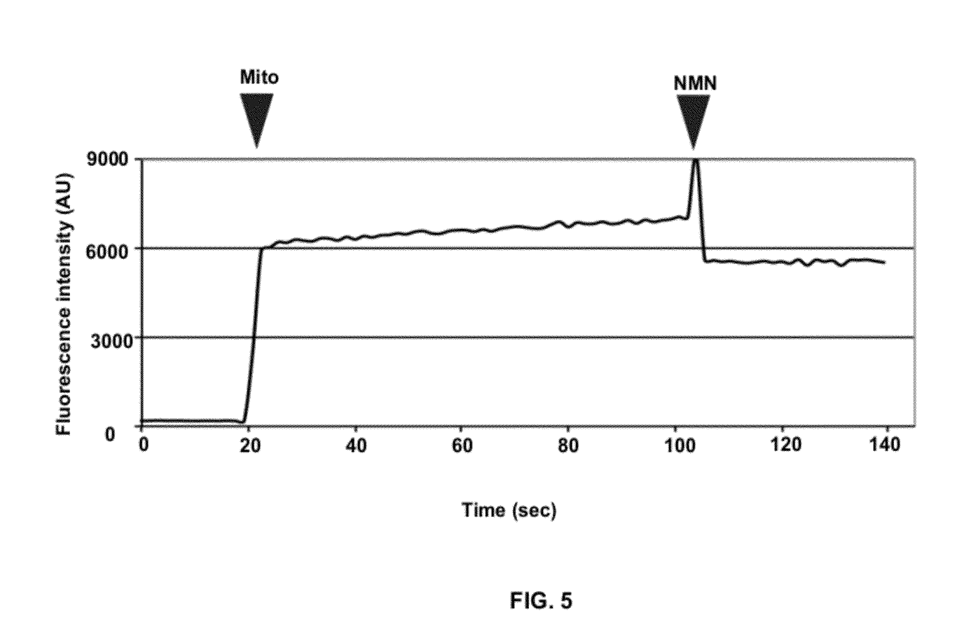

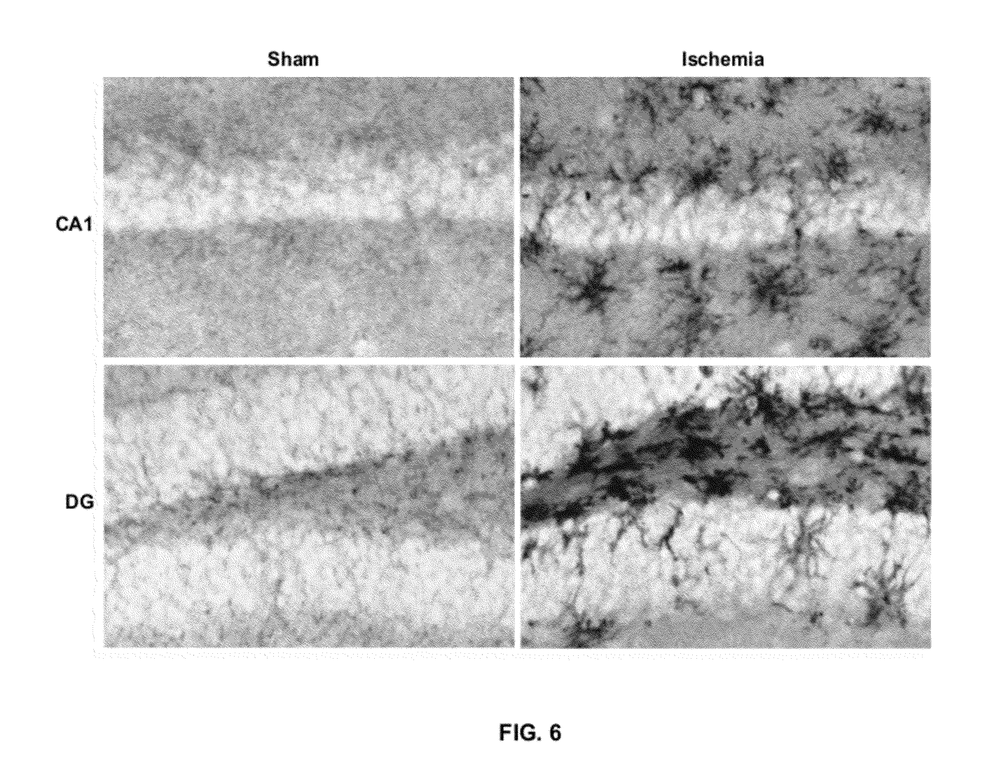

To determine whether mitochondrial NAD levels are affected, mitochondria and synaptosomes were isolated from post-ischemic hippocampal tissue and control sham operated animals. The total NAD(H) content was then determined by using cyclic enzymatic assay (FIG. 4A). As the data indicate there is a significant decrease in both mitochondrial and synaptosomal NAD(H) pools that is functionally reflected in lower spare respiratory capacity of post-ischemic synaptic mitochondria as determined by the Seahorse flux analyzer (FIG. 4B). Interestingly CD38 intracellulary also is localized in mitochondrial membranes (FIG. 5) and this way can catabolize cytosolic and mitochondrial NAD⁺. Thus increased CD38 activity can indirectly affect the mitochondrial functions.

To assess the cell-type specific changes in CD38 levels, immunostaining with CD38 antiserum was utilized (6). The CD38 immunoreactivity was very low in tissue of control animals whereas in post-ischemic animals there was a dramatic increase in the CD38 immunoreactivity, particularly in non-neuronal cells within the vulnerable hippocampal sub-regions (CA1 and DG) (FIG. 6) (6). To confirm that predominantly microglia are overexpressing CD38 a double staining technique with biotinylated tyramine amplification (28) for CD38 antigen can be used because the standard fluorescence staining did not visualize clearly CD38. These data suggests that the increase in CD38 expression in post-ischemic brain contributes, at least partly, to the higher NAD⁺ catabolic activity and consequently to the reduction of NAD(H) levels.

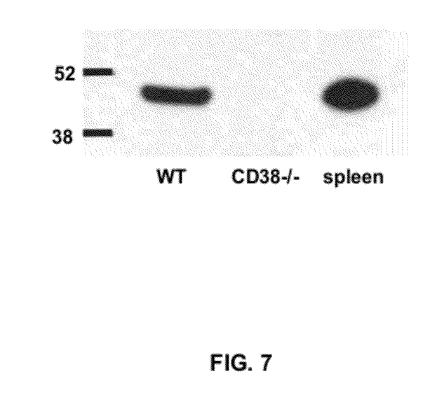

Since concomitantly the mitochondrial respiratory capacity is compromised (FIGS. 4A-4B) this data strongly suggest that the CD38 over-activation can, although indirectly, alter mitochondrial functions. Furthermore, the ischemia-induced changes in CD38 levels was assessed using western blots. These data confirmed a significant increase in CD38 expression particularly in the vulnerable hippocampal sub-regions (data not shown). CD38 knockout mice were obtained which are homozygous and CD38 expression in the brain tissue was not detectable by western blots (FIG. 7).

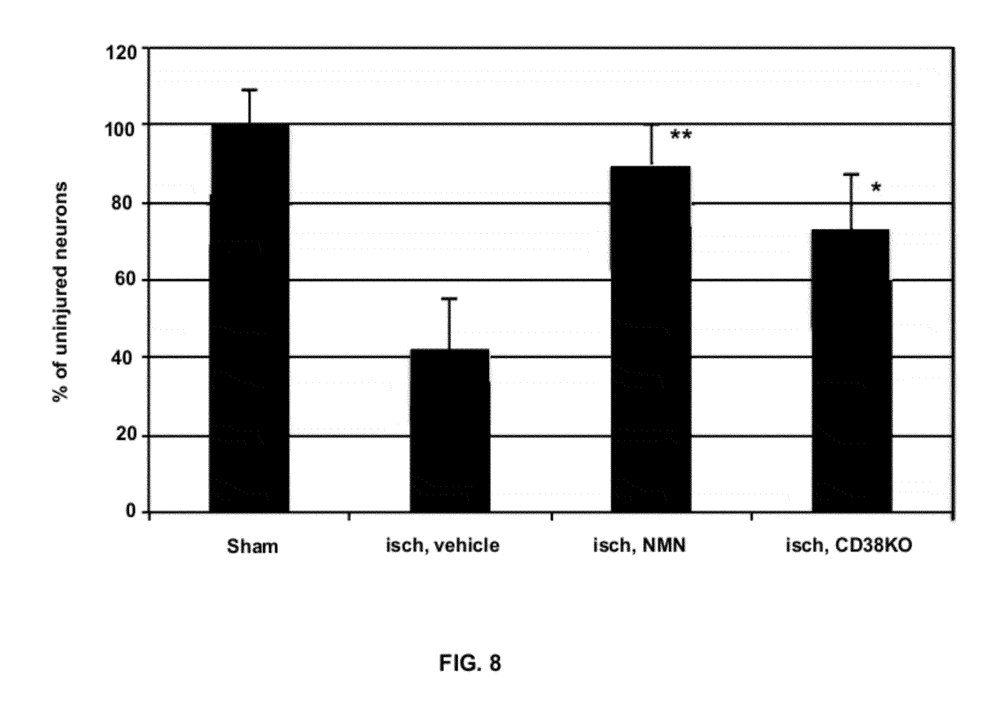

The CA1 sector of the hippocampus is the area of the brain vulnerable to the global cerebral ischemia. Wild type mice treated with vehicle or NMN and CD38-null mice are subjected to 10 min of global cerebral ischemia and the neuronal cell death in the CA1 sector of the hippocampus is determined following 6 days of recovery compared to control (sham operated mice). The percentage of uninjured neurons in wild type mice treated with nicotinamide mononucleotide and in CD38-null mice is significantly greater than wild type mice treated with vehicle only (FIG. 8).

Ischemic insult leads to dramatic reduction in NAD(H) levels and a significant increase in NAD⁺ glycohydrolase activity of the CD38 enzyme. The reduced NAD(H) pools result in compromised mitochondrial functions. Ischemia leads to marked increase in CD38 immunoreactivity, particularly in neuroglial cells and accelerated transformation of microglia into an activated form. These novel findings clearly stress the importance of utilizing the CD38-null transgenic animals as tools for revealing the role of CD38 in mechanisms leading to brain damage.

In summary, the present invention supports that CD38 activation and tissue NAD(H) depletion play a key role in mechanisms of cell death following acute brain insult. Furthermore, diabetes accelerates the post-ischemic microglial activation and post-ischemic nicotinamide mononucleotide administration reverses tissue NAD⁺ catabolism and dramatically ameliorates the ischemic cell death. This metabolically effective neuroprotection of nicotinamide mononucleotide could be translated to clinical trials and ultimately improve the neurologic outcome after stroke or traumatic brain injury.

### EXAMPLE 7

### Research Design and Methods

An object of the present invention is to establish the role of CD38-dependent cellular and mitochondrial NAD⁺ depletion and microglial activation as a key mechanism in the aggravation of ischemic brain damage in diabetic subjects. To achieve this, CD38 null mice are utilized and a state-of-the art approach that allows examination of mitochondrial functions in cell cultures and in isolated mitochondria and synaptosomes from mouse brain sub-regions. Finally, to prevent the fatal mitochondrial and cellular NAD⁺ depletion, animals are treated with precursors of the NAD⁺ salvage pathways and inhibitors of CD38 activity (NMN and NR). To visualize and quantify the relative NAD⁺ levels in brain tissue sections, a novel histoenzymatic staining technique is used that allows visualization of NAD(H) at the microscopic cellular and subcellular levels (1). There is a significant decrease in tissue NAD(H) levels concomitant with significant increased CD38 activity following forebrain ischemia. Furthermore, post-treatment of animals with nicotinamide mononucleotide at 1 hour of recovery almost completely eliminate the ischemic brain damage, suggesting a new avenue of neuroprotection for the treatment of acute brain injury. Once activated, CD38 can very rapidly consume tissue NAD⁺ pools and plays a key role in microglial activation (3). Thus, by inhibiting this enzyme and feeding into the NAD⁺ salvage pathway, one can significantly ameliorate the brain tissue damage following the ischemic insult in diabetic animals.

To show that CD38 significantly contributes to the NAD⁺ degradation, one can utilize CD38-null mice from Jackson Laboratories (B6.129-Cd38^{tm1Lnd}/J). The NADase activity in the plasma membrane and mitochondria is essentially absent in most tissues from CD38-deficient mice (6) and the CD38 protein is not detected in brain tissue by western blots. However, before examining the CD38 knockout animals the temporal, regional and cell-type specific changes in NAD(H) levels is determine following transient forebrain ischemia in diabetic mice. The diabetic condition is induced by streptozotocine (SPZ) injection and the animals with blood glucose levels over 300 mg/dl are considered diabetic. Diabetic mice 1 and 6 months following SPZ injection are used.

Diabetes causes weight loss and changes in other features that indicate the severity of diabetes. Therefore, the primary diabetic management of animals is to weigh them every week to determine if they have lost 20% of their weight. Animals that have lost >20% body weight are identified and excluded from experiments.

### EXAMPLE 8

### Post-ischemic Tissue and Mitochondrial NAD⁺ Hydrolysis and CD38 Activation

The present invention shows that 4 minute forebrain ischemia in diabetic animals leads to cell death by 48 h of reperfusion and the animals survive at least three days. Animals subjected to a 5 min or longer ischemic period died within 48 hours. In normoglycemic subjects following 4 min of forebrain ischemia no cell death was observed. Therefore, NAD(H) levels and CD38 activity is examined after 4 min forebrain ischemia at 3, 6, 16 and 24 h of recovery before the imminent cell death is observed as reflected by positive Fluoro Jade B staining. A dissection technique for fresh brain that allows separation of mouse cortex, striatum, and the individual sub-regions of the hippocampus. Aliquots of tissue homogenate are collected as well as isolate mitochondria and synaptosomes from these brain regions. The NAD(H) levels in these samples is determined utilizing a cyclic enzymatic assay with subnanomolar NAD(H) detection levels (13). In another set of animals, their brains are frozen and processed for tissue NAD(H) visualization in brain sections as described in (1). The CD38 activity is assessed by using 1-etheno-NAD (eNAD) (6). The mitochondrial NAD(H) depletion can be due to opening of the MPT pore (10), (13). Diabetes-induced hyperglycemia leads to profound acidosis during the ischemic insult and early recovery (15). Furthermore, acidosis promotes the MPT pore opening during the early reperfusion period (15), (16). This suggests that in diabetic animals the mitochondrial NAD⁺ depletion occurs during the first hours of recovery. To show that the mitochondrial NAD(H) pools are depleted, NAD(H) levels in isolated mitochondria are determined. During the mitochondrial isolation procedure, the permeability transition pore is closed due to the use of a high concentration of calcium chelating agent (1 mM EDTA) and low temperature (4° C.). Therefore, changes in the mitochondrial NAD(H) content induced in vivo due to MPT pore opening should be preserved (10). Similarly, NAD(H) levels in the synaptosomal fraction and tissue homogenate are determined. NAD⁺ in these samples are assessed by HPLC. As a control, sham operated diabetic animals are used. Furthermore, to examine whether diabetes is aggravating the post-ischemic NAD⁺ catabolism, the same ischemic insult is induced in normoglycemic animals and the temporal and regional changes in NAD⁺ levels are determined.

### EXAMPLE 9

### Effect of NAD⁺ Loss on Mitochondrial Respiration

Damage to mitochondrial membranes leads to significant reduction of matrix NAD(H) (10), (14), (17). The loss of mitochondrial NAD(H) should significantly inhibit respiration, particularly when supported by the complex I substrate (pyruvate). This is because pyruvate dehydrogenase (PDH) reduces NAD⁺ to NADH, which serves as an electron donor to complex I of the respiratory chain. To examine whether the NAD(H) depletion coincides with respiratory inhibition, mitochondrial and synaptosomal respiratory capacity is determined. Thus, at recovery times when a significant depletion of NAD(H) levels is observed, non-synaptic mitochondria and synaptosomes are isolated from the whole hippocampi and their respiratory capacity measured (18), (19). In experiments with synaptosomes, the spare respiratory capacity is determined as the difference between the control and maximum oxygen consumption rates (19). Synaptosomes are incubated in the presence of glucose plus pyruvate and the oxygen consumption rates are determined before and after oligomycin, and then FCCP additions. The basal control respiration of synaptosomes are measured, with the respiratory rates reflecting leakiness of the inner mitochondrial membrane to protons (oligomycin treatment) and the maximum respiratory rates (FCCP-stimulated oxygen consumption rates). The mitochondrial respiration are measured with both complex I- (pyruvate and malate) and complex II- (succinate) linked substrates. The state 3 (ADP-stimulated), state 4 (in the presence of oligomycin), and the maximum oxygen consumption rates (FCCP stimulated) is determined. Since the synaptosomal and mitochondrial protein yield from mouse hippocampi is around 0.2 mg, the Seahorse extracellular metabolic flux analyzer (XF24) is utilized to perform these experiments which allows measurement of respiration using only around 15 μg of synaptosomal protein and 2-3 μg of mitochondrial protein, respectively (18), (20). These experiments are performed in samples obtained at every recovery period, and non-ischemic, sham-operated diabetic animals are used as controls. To confirm that the inhibition of mitochondrial or synaptosomal respiration is due to the NAD⁺ degradation, synaptosomes isolated from post-ischemic tissue are incubated with 10 mM NAD⁺ during the preparation procedure for respiration measurements with the XF24. Incubating synaptosomes with NAD⁺ increases the synaptosomal NAD(H) levels 2-fold when compared to control samples. This uptake of NAD⁺ by synaptosomes was blocked by the purinergic receptor P2X(7)-gated channels inhibitor brilliant blue, suggesting that increased synaptosomal NAD(H) content was not due to a contamination. The respiratory rates of post-ischemic mitochondria are improved by adding NAD⁺ into the respiration medium (21). Thus, synaptosomes isolated from post-ischemic tissue and incubated with NAD⁺ should show improved respiratory rates suggesting that the main cause of the post-ischemic reduced respiratory capacity of synaptosomes is significant reduction of NAD(H) levels.

Mitochondrial NAD(H) levels are at least partially reduced after the first hour of reperfusion presumably due to transient activation of the MPT. At later recovery periods, tissue NAD(H) decrease since the NAD⁺ translocates from mitochondria to the cytosol and is hydrolyzed by the activated NAD⁺ glycohydrolases CD38 and PARP1. CD38-dependent overactivation of microglia under diabetic conditions leads to aggravation of ischemic brain damage, astrocytes and neurons prepared from CD38 knockout animals with microglia (9) from wild type or CD38-null animals are co-cultured. The co-cultures are then subjected to OGD and the cell death assessed at 24 h of recovery.

Exposure of cortical astrocytes to OGD plus ISS for 4 h results in significant (>50%) cell death after 24 h of reoxygenation (24) was determined. When cortical neurons are subjected to the same insult for 1 h, a similar level of cell death (40-50%) was observed. Cells cultured under hyperglycemic conditions (30 mM glucose) are more sensitive to the oxygen-glucose deprivation (OGD) insult. The CD38-deficient cells will have elevated basal levels of NAD(H) since the brain NAD(H) levels in CD38-null mice are several-fold higher when compared to wild type mice (6). The protective effect of CD38 knockout in specific cell types will also depend on the oxygen-glucose deprivation-induced loss of mitochondrial NAD(H) that can promote total cellular NAD(H) depletion. Since the majority of NAD(H) is localized in mitochondria (25), a dramatic depletion of cellular NAD(H) must be accompanied by loss of NAD(H) from the mitochondrial matrix that was consequently degraded. Therefore, the changes in NAD(H) content in control cells and cells subjected to OGD followed by 1, 6 and 24 h of recovery are examined. NAD(H) degradation is significantly inhibited in CD38-deficient cells following oxygen-glucose deprivation when compared to control cell culture. Furthermore, maintaining physiologic NAD(H) levels improves the mitochondrial respiratory functions and increases cell survival after the oxygen-glucose deprivation. The less efficient protective effect of NMN treatment with CD38-deficient cells when compared to the PARP-1 inhibitors 3-AB or PJ34 suggest that NMN specifically inhibits CD38.

The co-culture experiments reveal the contribution of CD38 activated microglia to OGD-induced cell death. Since microglia are resistant to oxygen-glucose deprivation conditions for up to 8 hours (9), one would not expect that these cells will contribute to cell death assessment. CD38 deficient microglia cause less aggravation of cell death in co-cultures when compared to microglia from wild type animals or cell death is less profound in pure neuronal or astrocytic cultures when compared to co-cultures with microglia (9).

### EXAMPLE 10

### Effect of CD38 Knockout on Microglial Activation and Ischemic Brain Damage

Diabetes is associated with systemic inflammation reflected in increased levels of circulating pro-inflammatory cytokines (22). CD38 is an ectoenzyme that participates in a systemic inflammatory response by activating immune cells including resident immune cells in the brain, microglia. Early activation of microglia in diabetic animals following ischemic insult plays a crucial role as an aggravating factor in ischemic brain damage. CD38-null mice are utilized to examine the effect of this enzyme activity on microglial activation and ischemic brain injury. Diabetic CD38-null animals show less pronounced reduction in NAD(H) levels when compared to control, wild type diabetic animals at 24 h recovery period. Consequently, NAD(H) visualization by a histoenzymatic technique in these mice reveals higher NAD(H) levels in CD38-deficient animals at 24 h reperfusion when compared to control animals. Higher NAD(H) levels in post-ischemic tissue result in amelioration of the ischemia-induced damage.

### EXAMPLE 11

### Protective Effect of Nicotinamide Mononucleotide Against Ischemic Brain Damage in Diabetic Animals

NAD⁺ glycohydrolase ADP-ribosyl cyclase CD38 can significantly contribute to NAD⁺ degradation following ischemic insult and increase the post-ischemic activation of microglia. This is supported by the data showing that treatment of brain tissue with nicotinamide mononucleotide, an inhibitor of CD38, prevents tissue NAD⁺ degradation. Additionally, CD38 enzyme activity is significantly increased concomitant with reduced NAD⁺ levels following ischemic insult. Since nicotinamide mononucleotide not only inhibits NAD⁺ catabolism (12) but also serves as a precursor in the NAD⁺ salvage pathway (4) and can inhibit microglial activation, this compound will have a potent, therapeutic effect and improve outcome following ischemic insult in diabetic animals. In experiments, nicotinamide mononucleotide was administered at a dose of 500 mg/kg to post-ischemic animals at 1 h after onset of reperfusion that resulted in dramatic amelioration of cell death in the CA1 sector of the hippocampus. Post-ischemic animals that received nicotinamide mononucleotide showed complete recovery of brain tissue NAD(H) levels. These results clearly suggest that nicotinamide mononucleotide administration is much more efficient in treatment of ischemic brain damage when compared to studies utilizing PARP-1 inhibitors or PARP^{-/-}transgenic animals where the NAD(H) levels were increased only to about 60% of control (26). A 500 mg/kg dose of nicotinamide mononucleotide administered intraperitoneally provides significant protection in normoglycemic animals.

The present invention demonstrates the therapeutic effect of nicotinamide mononucleotide administration on post-traumatic chronic neuroinflammation and associated neurodegeneration in a clinically relevant mouse model of traumatic brain injury. An object of the present invention is to develop an efficient NMN treatment strategy that reduces post-traumatic tissue loss and neurodegeneration, and reduces the long-term functional deficits associated with traumatic brain injury. The present invention demonstrates chronic neurodegeneration and microglial activation up to 12 months post-injury in an experimental model of traumatic brain injury and that nicotinamide mononucleotide treatment is highly neuroprotective in a model of ischemic brain injury and results in improved functional recovery following experimental traumatic brain injury.

### EXAMPLE 12

### TBI Induces Progressive Tissue Damage and Neurodeqeneration up to 1 Year Post-injury

In a clinically relevant model of traumatic brain injury, controlled cortical impact (CCI) (Fox et al. 1998), was induced in C57Bl/6 mice and these animals were followed for up to 1 year post-injury to assess post-traumatic tissue loss and neurodegeneration. A moderate level injury was induced by an impact velocity of 6 m/sec and a cortical tissue deformation depth of 2 mm. Sham injured animals (craniotomy but no impact) served as controls. Repeated high field (7-tesla) T2-weighted magnetic resonance imaging (MRI) measurements of lesion volume were performed on two groups of mice subjected to moderate-level CCI injury. One group was evaluated for changes between 24 h and 3 months post-injury; a second group was used to compare changes in TBI-induced lesion volume between 6 and 12 months. This longitudinal analysis demonstrated that in both cohorts of animals there were progressive lesion volume increases over time, with a significant increase in lesion size at 12 months post-injury when compared to the measurement from the same animal at 6 months post-injury. T2-weighted MRI images from a traumatic brain injury mouse imaged at 6 and 12 months post-injury show that the lesion (hyperintense area) expands laterally over time through the hippocampus and into the lateral ventricles. The mice were then euthanized at 12 months post-injury and perfusion-fixed them with paraformaldehyde for histological assessment of neuronal cell loss in the hippocampus using unbiased stereological techniques. There was significant neurodegeneration in the 12 month injured tissue with significant reduction of hippocampal neurons in the CA1, CA3 and dentate gyrus (DG) sub-fields when compared to age matched sham-injured controls.

Tissue at 12 months post-injury also showed increased protein oxidation in the injured cortex as well as significantly increased expression of GFAP and lba-1, markers of activated astrocytes and microglia respectively, in the injured cortex when compared to age-matched control tissue as measured by Western immunoblotting. lba-1 immunohistochemistry was performed to label microglia in the 12 month injured brain. Based on cell morphological features, microglia can be classified into three categories corresponding to increasing activation status: ramified (resting), hypertrophic, and bushy. Ramified microglia have small cell bodies and thin, long and highly branched processes. In contrast, hypertrophic microglia have larger cell bodies with thicker, shorter and highly branched processes, whereas bushy microglia have multiple short processes that form thick bundles around enlarged cell bodies. CNS injuries, such as stroke and TBI, cause transformation of resting ramified microglia into more active phenotypes, such as hypertrophic and bushy forms. An unbiased stereological assessment of microglial cell number and activation phenotype in the injured cortex of the 12 month injured mice was performed. The data revealed increased numbers of activated microglia at 12 months post-injury, as reflected by hypertrophic and bushy morphological phenotypes when compared to age-matched controls. In addition, there was a trend to a decrease in the numbers of ramified microglia in the 12 month injured samples suggesting fewer resting microglia in the injured samples. These data suggest that chronic microglial activation persists for up to 1 year following experimental traumatic brain injury and are consistent with other animal and clinical studies indicating that post-traumatic neuroinflammation persists for months to years after traumatic brain injury.

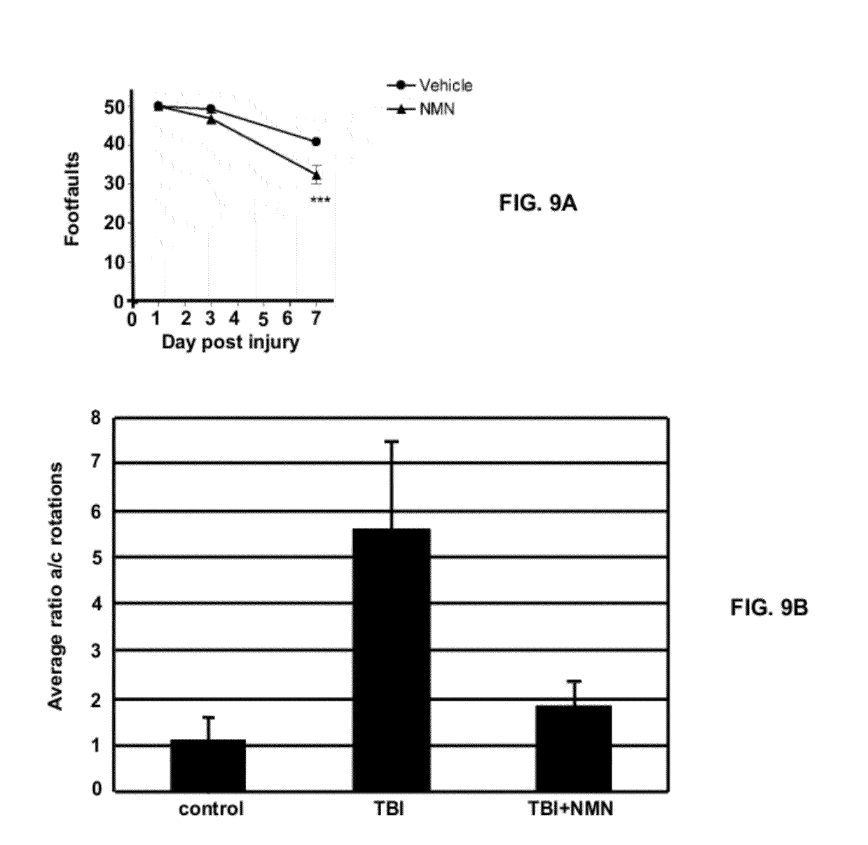

The effect of nicotinamide mononucleotide on traumatic brain injury-induced changes in fine motor coordination examined using a beam walking task in which mice were trained to cross an elevated narrow wooden beam. The mouse was placed on the left end of the beam and the number of foot faults for the right hind limb was recorded over 50 steps on the beam. A basal level of motor function was achieved following 3 days training prior to surgery with an acceptance level of less than 10 faults per 50 steps. The beam walk test was performed at day 1, 3 and 7 after traumatic brain injury and nicotinamide mononucleotide treated traumatic brain injury mice showed a significant improvement in motor performance at 7 days post-injury when compared to the vehicle-treated traumatic brain injury mice (FIG. 9A, p<0.001). Furthermore, traumatic brain injury mice performed an open field test at 7 days post-injury. The animals were placed in a translucent polypropylene box in a quiet room with dimmed lighting. Their activity was measured and recorded using the Any-maze video tracking system. The total distance traveled by the animal was recorded during a 15 minute testing period with 3 minute consecutive intervals. Their maximum and medium speed, clockwise (C), and anti-clockwise (A) turns, total number of turns and absolute turn was recorded. The traumatic brain injury induced insult did not affect the animals overall exploratory activity or any other measured parameters. The animals did preferentially turn anti-clockwise during the recorded period, as indicated by the increase in the A/C ratio in the vehicle treated group (FIG. 9B). The non-injured animals turned randomly right or left during the open field test that is reflected in A/C rotations ratios equal to one.

Thus, vehicle-treated traumatic brain injury mice showed deficits in cognitive function as reflected by an increase in the anti-clockwise rotations in the open field test. Notably, NMN-treated traumatic brain injury mice inclined to rotate in both directions randomly and were virtually indistinguishable from the naïve mice (non-injured controls). These data suggest that NMN-treatment at 1 hour post-injury improves motor and cognitive function recovery after moderate-level traumatic brain injury.

The following references are cited herein:
1. Balan et al. Brain Res, 2010, 1316C:112-119.
2. Gray et al., Lancet Neurol, 2007, 6(5):397-406.
3. Mayo et al., J Immunol, 2008, 181(1):92-103.
4. Belenky et al. Trends Biochem Sci, 2007, 32(1):12-9.
5. Kristian et al., Journal of Neuroscience research, 2011, 89:1946-55.
6. Aksoy et al., Biochem Biophys Res Commun, 2006, 345(4):1386-92.
7. Szabo, C. and V. L. Dawson, Trends Pharmacol Sci, 1998, 19(7):287-98.
8. Chidambaram, N. and C. F. Chang, Int J Biochem Cell Biol, 1998, 30(9):1011-8.
9. Yenari et al., Stroke, 2006. 37(4):087-93.
10. Di Lisa et al., J Biol Chem, 2001, 276(4):2571-5.
11. Ruddick et al., Expert Rev Mol Med, 2006. 8(20):1-27.
12. Formentini et al., Biochem Pharmacol, 2009, 77(10):1612-20.
13. Kellenberger et al., Bioorg Med Chem Lett. 2011, 21(13):3939-42.
14. Kristian, T. and G. Fiskum, Brain Res Brain Res Protoc, 2004, 13(3):176-82.
15. Kristian, T., Cell Calcium, 2004. 36(3-4):221-33.
16. Kristian et al., J Neurotrauma, 2001,18(10):1059-74.
17. Barsoum et al., Embo J, 2006, 25(16):3900-11.
18. Gerencser, A. A., et al., Anal Chem, 2009, 81(16):6868-78.
19. Choi et al., J Neurochem, 2009, 109(4):1179-91.
20. Cho et al., Science, 2009, 324(5923):102-5.
21. Fiskum et al., Ann N Y Acad Sci, 2008, 1147:129-38.
22. Wierusz-Wysocka et al., J Leukoc Biol, 1987, 42(5):519-23.
23. Hazelton, J. L., et al., J Neurotrauma, 2010, 27(4):753-62.
24. Danilov, C. A. and G. Fiskum, Glia, 2008, 56(7):801-8.
25. Mayevsky, A. and G. Rogatsky, Am J Physiol Cell Physiol, 2007, 292(2):C615-40.
26. Endres et al., J Cereb Blood Flow Metab, 1997, 17(11):1143-51.
27. Vereczki et al., J Cereb Blood Flow Metab, 2006, 26(6):821-35.
28. Hazelton et al., J Neurosci Res 2009, 87(5):1250-9.

What is claimed is:

1. A method of treating a mammal having a neuropathophysiological condition, comprising the step of:
administering to the mammal in need of such treatment a compound selected from nicotinamide or salts or prodrugs thereof, nicotinamide mononucleotide or salts or prodrugs thereof, nicotinamide adenine dinucleotide or salts or prodrugs thereof, nicotinamide riboside or salts or prodrugs thereof, phosphoribosyltransferase, or combinations thereof.

2. The method of claim 1, wherein the compound is nicotinamide or salts or prodrugs thereof, nicotinamide mononucleotide, nicotinamide adenine dinucleotide, nicotinamide phosphoribosyltransferase, or combinations thereof.

3. The method of claim 1, wherein the compound is a polypeptide capable of catalyzing the conversion of nicotinamide to nicotinamide mononucleotide.

4. The method of claim 1, wherein the condition is selected from the group consisting of traumatic brain injury, ischemia, Alzheimer's Disease, multiple sclerosis, Amyotrophyc lateral sclerosis and peripheral neuropathy.

5. The method of claim 1, wherein said compound is nicotinamide mononucleotide administered in a dose of from about 10 mg/kg to about 1000 mg/kg.

6. The method of claim 1, wherein said mammal is a human.

7. A method for treating a mammal having a neuropathophysiological condition or suspected to develop said neuropathophysiological condition, comprising the step of:
administering to said mammal an inhibitor of CD38 NAD⁺ glycohydrolase activity.

8. The method of claim 7, wherein said inhibitor is an anti-CD38 antibody.

9. The method of claim 7, wherein said inhibitor is a compound selected from nicotinamide or salts or prodrugs thereof, nicotinamide mononucleotide or salts or prodrugs thereof, nicotinamide adenine dinucleotide or salts or prodrugs thereof, nicotinamide riboside or salts or prodrugs thereof, nicotinamide phosphoribosyltransferase, or combinations thereof.

10. The method of claim 7, wherein said inhibitor decreases CD38 expression.

11. The method of claim 10, wherein said inhibitor is an siRNA molecule, an antisense oligonucleotide, or a peptide nucleic acid.

12. The method of claim 7, wherein the condition is selected from the group consisting of traumatic brain injury, ischemia, Alzheimer's Disease, multiple sclerosis, Amyotrophyc lateral sclerosis and peripheral neuropathy.

13. The method of claim 7, wherein said compound is nicotinamide mononucleotide administered in a dose of from about 10 mg/kg to about 1000_mg/kg.

14. The method of claim 7, wherein said mammal is a human.

15. A method for identifying an agent for treating a neuropathophysiological condition, wherein said method comprises: (a) determining whether or not a test agent inhibits CD38, wherein inhibition of CD38 indicates that said test agent is a candidate agent, and (b) administering said candidate agent to a mammal to determine whether or not said candidate agent treats the neuropathophysiological condition in said mammal, wherein an improvement in symptoms of said neuropathophysiological condition indicates that said candidate agent is said treatment agent.

16. The method of claim 15, wherein said step (a) comprises using an in vitro CD38 activity assay.

17. The method of claim 15, wherein said mammal is a mouse.
